(19) United States
(12) Patent Application Publication
(10) Pub. No.: US 2025/0361468 A1
(43) Pub. Date: Nov. 27, 2025

(54) COMBINATION OF METHANOL LOOP AND BIOGAS PRODUCING UNIT

(71) Applicant: Topsoe A/S

(72) Inventors: Martin ØSTBERG, Tune (DK); Peter Mølgaard MORTENSEN, Roskilde (DK); Troels Juel FRIIS-CHRISTENSEN, Smørum (DK)

(73) Assignee: Topsoe A/S, Kgs. Lyngby (DK)

(21) Appl. No.: 18/874,665

(22) PCT Filed: Jun. 15, 2023
(86) PCT No.: PCT/EP2023/066153

§ 371 Date: Dec. 13, 2024

(30) Foreign Application Priority Data
Jun. 17, 2022 (EP) 22179658.4

(51) Int. Cl. C12M 1/107; C01B 3/38; C12M 1/00; C12M 1/02

(52) U.S. Cl. CPC C12M 21/04; C01B 3/38; C12M 41/18; C12M 43/02; C12M 47/18; C01B 2203/0233; C01B 2203/0283; C01B 2203/061; C01B 2203/0883; C01B 2203/0894

(57) ABSTRACT

A plant, in particular a methanol plant, is provided, said plant comprising: a first biomass feed, a biomass digester, arranged to receive the first biomass feed and convert it to a biogas stream, a reformer section arranged to receive at least a portion of the biogas stream from the biomass digester and provide a first synthesis gas stream, a synthesis section, arranged to receive a synthesis gas stream from the reformer section and provide a raw product stream; and a first hydrocarbon-containing off-gas stream, and a distillation section arranged to receive at least a portion of the raw product stream and provide at least an upgraded product stream and a second hydrocarbon-containing off-gas stream. At least a portion of said first and/or at least a portion of said second off-gas stream is arranged to be recycled as additional feed to the biomass digester. A process using said plant is also described.

### TECHNICAL FIELD

The present invention relates to a chemical plant and process for effective use of biogas, in which carbon utilisation can be increased.

### BACKGROUND

Biogas is a renewable energy source that can be used for heating, electricity, and many other operations. Biogas can be cleaned and upgraded to natural gas standards, to become bio-methane. Biogas is considered to be a renewable resource because its production-and-use cycle is continuous, and it generates no net carbon dioxide. When the organic material has grown, it is converted and used. It then regrows in a continually repeating cycle. From a carbon perspective, as much carbon dioxide is absorbed from the atmosphere in the growth of the primary bio-resource as is released, when the material is ultimately converted to energy.

Biogas is a mixture of gases produced by the breakdown of organic matter in the absence of oxygen. Biogas can be produced from raw materials such as agricultural waste, manure, municipal waste, plant material, sewage, green waste or food waste. Biogas is primarily methane (CH₄) and carbon dioxide (CO₂) and may include small amounts of hydrogen sulfide (H₂S), moisture, siloxanes, and possibly other components.

A biogas contains typically about 50-60% methane and 40-50% CO₂. To utilize the CO₂ in the biogas, it is advantage to produce a syngas that can be fed to a downstream synthesis that takes advantage of the H₂/CO ratio that can be obtained. One such synthesis is a methanol (MeOH) synthesis where methanol is produced from the synthesis gas in a methanol loop. Alternatively, FT synthesis, a gasoline (TIGAS) synthesis or an acetic acid synthesis could be used.

A process and plant for converting biogas to methanol is described in WO2020254121.

It would be desirable to provide chemical plants for effective use of biogas. In particular, there is the potential to recycle any organic materials such as hydrocarbons present in off gas streams in a chemical plant, thus increasing the carbon efficiency of the plant. Furthermore, the digester operates at an optimal temperature of about 50° C., making heat supply to the digester a major cost for a plant in which biogas production takes place. Recycling of various off-gas streams from the chemical plant can assist in providing heat to the digester, as well as improving the carbon utilisation.

### SUMMARY

The bacteria which convert biomass feed into biogas are capable of digesting most hydrocarbon feedstocks. This has value when combining a biogas unit with a chemical synthesis unit, as it has been discovered by the present inventors that various hydrocarbon-containing off-gas and purge gas streams can be recycled and fed into the biomass digester as additional feed.

A plant, in particular a methanol plant, is therefore provided, said plant comprising:
a first biomass feed,
a biomass digester, arranged to receive the first biomass feed and convert it to a biogas stream,
a reformer section arranged to receive at least a portion of the biogas stream from the biomass digester and provide a first synthesis gas stream,
a synthesis section, arranged to receive a synthesis gas stream from the reformer section and provide a raw product stream; and a first hydrocarbon-containing off-gas stream,
a distillation section arranged to receive at least a portion of the raw product stream and provide at least an upgraded product stream and a second hydrocarbon-containing off-gas stream,
wherein at least a portion of said first and/or at least a portion of said second off-gas stream is arranged to be recycled as additional feed to the biomass digester.

A process for providing a product stream from a first biomass feed, in a plant as described herein, said process comprising:
feeding a first biomass feed to a biomass digester, and converting it to a biogas stream,
feeding at least a portion of the biogas stream from the biomass digester to the reformer section so as to provide a first synthesis gas stream,
feeding a synthesis gas stream from the reformer section to the synthesis section so as to provide a raw product stream; and a first hydrocarbon-containing off-gas stream,
feeding at least a portion of the raw product stream to a distillation section so as to provide at least an upgraded product stream and a second hydrocarbon-containing off-gas stream, and;
recycling at least a portion of said first and/or at least a portion of said second off-gas stream as additional feed to the biomass digester.

Further details of the technology are provided in the enclosed dependent claims, figures and examples.

### LEGENDS TO THE FIGURES

The technology is illustrated by means of the following schematic illustrations, in which:

FIG. 1 shows a schematic process layout of the plant of the invention.

FIG. 2 shows a schematic process layout for a plant, using electrical reforming of biogas to produce methanol.

FIG. 3 shows a schematic process layout of a distillation section suitable for upgrading the raw product stream.

### DETAILED DISCLOSURE

Unless otherwise specified, any given percentages for gas content are % by volume. All feeds are preheated as required.

The term “synthesis gas” is meant to denote a gas comprising hydrogen, carbon monoxide and also carbon dioxide and small amounts of other gasses, such as argon, nitrogen, methane, etc.

In the following a “waste water” stream is a stream comprising a majority (i.e. more than 50% by volume) of water. The waste water stream(s) may be liquid or gaseous streams, but are—in a preferred embodiment—liquid.

An “off-gas” stream is a stream produced in a plant, which is a mixture of a number of components. Off-gas streams are produced as the by-product of a chemical or physical process, and are not the primary streams of interest in the plant. Among other things, off-gases prevent build-up of inert species. Often off-gases are used as fuel, or flared off.

In a plant, where synthesis gas is produced and this is converted into e.g. MeOH in a MeOH loop, there are some off-gas streams and purge gas streams comprising hydrocarbons. These streams include e.g.:
The purge gas stream from the methanol loop that should ensure that inert gases is not accumulated. This stream is taken after the high pressure separator separating the methanol product from the unconverted gas fraction before it enters the recycle compressor.
The off-gas after the low pressure separator forming an additional gas fraction after de-pressuring the liquid methanol product in the high-pressure separator.
The collected off-gas from the vent wash column and the gas fraction from the stabilizer column, where the first fractionation (distillation) of the raw methanol is performed removed the gaseous species dissolved in the raw methanol. This is in a large extent carbon dioxide and methane, but will also contain methanol corresponding to the saturation at the cooling temperature.

These streams add up to about 5% of the total feed being a significant amount to recycle into the system. These gas streams can be recycled back to the digester, which then will reuse unconverted carbon dioxide and methane and consume other hydrocarbons by the bacteria. One challenge is a possible build-up of inert gases primarily nitrogen but possibly also argon. If this occurs, it may be necessary to take a fraction of the recycled gas and utilize this as a fuel for heating to balance the concentration of inert gases in the biogas.

As is indicated, the recycle of the off-gases and the purge gas may improve the overall utilization of the feed by about 4-5%.

A chemical plant is thus provided, which converts biomass feed to a product stream. In general terms, the plant comprises:
a first biomass feed,
a biomass digester,
a reformer section
a synthesis section and
a distillation section.

These components, their arrangement and their function will be discussed in detail in the following.

### Biomass Feed

A biomass feed is typically a liquid slurry, with a total solids content of between 20-40%. Apart from water, biomass principally comprises organic material which can be converted by the action of microbes to a biogas, e.g. in an anaerobic digestion with anaerobic organisms or methanogen inside an anaerobic digester. Sources of biomass feed include agricultural waste, such as manure, sewage, green waste and food waste, as well as industrial waste e.g. from food or drink production.

Apparatus for handling and supply of the biomass feed to the plant are known to the skilled engineer.

### Biomass Digester

A biomass digester is arranged to receive the first biomass feed and provide a biogas stream. The term “biogas” in connection with the present invention denotes a gas with the following composition:

| Compound | % |
|---|---|
| Methane | 50-75 |
| Carbon dioxide | 25-50 |
| Nitrogen | 0-10 |
| Hydrogen | 0-1 |
| Oxygen | 0-1 |

The bacteria which convert the biomass feed into biogas are capable of digesting most hydrocarbon feedstocks. This is important in the combination of a biogas unit with a chemical synthesis unit.

A biomass digester is typically in the form of a pressure reaction vessel with appropriate inlet(s) for biomass and outlet(s) for biogas. Additional inlets and outlets may be provided for the various waste water streams recycled according to the invention. Inlets and outlets may also be provided for e.g. sampling the contents of the digester or introducing or removing microbial matter.

The biomass digester operates most effectively at around 50° C. In one aspect, the plant comprises means for heating the biomass digester, preferably a heat exchanger.

In one aspect, at least a portion of the first and/or at least a portion of the second off-gas stream, or a combination of the first and second off-gas streams, is arranged to be fed through said heat exchanger, thereby heating the biomass digester. This makes effective use of heat energy in the off-gas streams.

Additionally, the reformer section and/or the synthesis section may comprise one or more heat exchangers, arranged to exchange heat between one or more cooling streams in said plant and one or more streams in said reformer section and/or said synthesis section; and thus provide one or more heated streams from said cooling streams, and wherein at least a portion of said heated stream(s) is arranged to heat the biomass digester. In this manner, off-gas streams may be used to heat the reformer section and/or the synthesis section (which may have a high heat requirement) before they are sent (at a lower temperature) to the biomass digester.

Compared to a non-heated biomass digester, a heated biomass digester provides a lower residence time in the vessel, and therefore a high production.

Direct heating with steam has the disadvantage of requiring an elaborate steam-generating system (including desalination and ion exchange as water pre-treatment) and can also cause local overheating. The high cost may only be justifiable for large-scale sewage treatment facilities. The injection of hot water raises the water content of the slurry and should only be practiced if such dilution is necessary.

Indirect heating is accomplished with heat exchangers located either inside or outside of the digester, depending on the shape of the vessel, the type of substrate used, and the nature of the operating mode.
1. Floor heating systems have not served well in the past, because the accumulation of sediment gradually hampers the transfer of heat.
2. In-vessel heat exchangers are a good solution from the standpoint of heat transfer as long as they are able to withstand the mechanical stress caused by the mixer, circulating pump, etc. The larger the heat-exchange surface, the more uniformly heat distribution can be effected which is better for the biological process.
3. On-vessel heat exchangers with the heat conductors located in or on the vessel walls are inferior to in-vessel-exchangers as far as heat-transfer efficiency is concerned, since too much heat is lost to the surroundings. On the other hand, practically the entire wall area of the vessel can be used as a heat-transfer surface, and there are no obstructions in the vessel to impede the flow of slurry.
4. Ex-vessel heat exchangers offer the advantage of easy access for cleaning and maintenance.

Further components and design of the biomass digester are known to the skilled engineer.

### Reformer Section

A reformer section is arranged to receive at least a portion of the biogas stream and provide a first synthesis gas stream.

The first synthesis gas stream typically comprises (in % by volume)
0.5-5% methane (dry)
40-70% H₂ (dry)
10-30% CO (dry)
2-20% CO₂ (dry)

The reformer section may comprise one or more of an autothermal reforming (ATR) unit, a steam methane reforming (SMR) unit and an electrically heated steam methane reforming (e-SMR) unit, and is preferably an electrically heated steam methane reforming (e-SMR) unit. Details of an e-SMR unit that is preferably used in the reformer section are found in WO2020254121.

Additional feeds (e.g. a steam feed or oxygen-rich feed) are supplied to the reformer section, as required, depending on the type of reforming to be carried out. For instance, SMR requires a steam feed, while ATR requires a steam feed and an oxygen-rich feed.

A first waste water stream is typically also provided by the reformer section.

### Synthesis Section

The synthesis section is arranged to receive a synthesis gas stream from the reformer section and provide a raw product stream, and a first hydrocarbon-containing off-gas stream.

The first hydrocarbon-containing off-gas stream typically has the following composition:
50-60% H₂,
25-30% CH₄,
4-7% CO₂,
3-5% CO,
0.2-1% CH₃OH,
and <100 ppm higher alcohols, <100 ppm by products, N₂ dependent on inlet N₂ (1-5%), <100 ppm H₂O

In one preferred embodiment, the synthesis section is a methanol synthesis section and the raw product stream is a raw methanol stream.

By the term “methanol synthesis section” is understood one or several reactors configured to convert synthesis gas into methanol. Such reactors can for example be a boiling water reactor, an adiabatic reactor, a condensing methanol reactor or a gas-cooled reactor. Moreover, these reactors could be many parallel reactor shells and sequential reactor shells with intermediate heat exchange and/or product condensation. It is understood that the methanol synthesis unit also contains equipment for recycling and pressurizing syngas feed to the methanol reactor(s). All constituents of the reformer feed stream are pressurized, either separately or jointly, upstream the re-forming reactor. Typically, steam is pressurized separately, whilst the other constituents of the reformer feed stream may be pressurized jointly. The pressure(s) of the constituents of the reformer feed stream is/are chosen so that the pressure within the reforming reactor lies between 5 to 100 bar, preferably between 20 and 40 bar, or preferably between 70 and 90 bar.

Suitably, the methanol synthesis section comprises:
a methanol reactor arranged to receive the synthesis gas stream from the syngas section and the second recycle stream from the first separator and provide a first methanol stream,
a high-pressure separator arranged to receive the first methanol stream from the methanol reactor and separate it into a second methanol stream and a first recycle stream,
wherein the first recycle stream from the high-pressure separator is arranged to be split in a first separator into a second recycle stream and a first purge stream,
wherein the second recycle stream is arranged to be compressed and mixed with the synthesis gas stream to the methanol reactor,
and wherein the first purge stream is arranged to be split in a second separator into a recycle hydrogen stream and a second purge stream, and wherein second purge stream is recycled as additional feed to the biomass digester.

In this embodiment of the plant, the methanol synthesis section may further comprise a low-pressure separator arranged to receive the second methanol stream from the high-pressure separator and provide a raw methanol stream and a third off-gas stream, and wherein the third off-gas stream is arranged to be recycled as additional feed to the biomass digester.

In this embodiment, the module

$$M = \frac{{H}_{2} - C {O}_{2}}{C O + C {O}_{2}} .$$

of the synthesis gas fed to the methanol synthesis section is typically in the range of 1.5 to 2.5.

In an alternative embodiment, the synthesis section is a Fischer-Tropsch (F-T) synthesis section and the raw product stream is a raw hydrocarbon stream. In this embodiment, the synthesis gas composition should have an H₂/CO ratio slightly above 2, where the exact value depends on the choice of FT catalyst.

There are at least three ways to adjust the syngas composition to match the module M or the H2/CO ratio required for a FT synthesis.
CO₂ can be removed upstream the reformer.
Additional methane could be added (if available).
Hydrogen could be added—this will typical be downstream the reformer, but could also be upstream.

### Distillation Section

A distillation section is arranged to receive at least a portion of the raw product stream and provide at least an upgraded product stream and a second hydrocarbon-containing off-gas stream.

The second hydrocarbon-containing off-gas stream typically has the following composition: 70-80% CO₂, 5-15% CH₄, 6-10% CH₃OH, 2-3% byproducts, 1-2% H₂, 0-0.5% CO, <1% N₂, and <10 ppm higher alcohols.

Various layouts of the distillation section are possible. In one aspect, the distillation section comprises a vent wash column, wherein said vent wash column is arranged to receive at least a portion of the raw product stream from the synthesis section and provide at least a first upgraded product stream and a vent column off-gas stream, wherein at least a portion of said vent column off-gas stream is arranged to be recycled as additional feed to the biomass digester.

Furthermore, the distillation section suitably comprises a stabilizer column, wherein said stabiliser column is arranged to receive at least a portion of the first upgraded product stream from the vent wash column, and provide at least a second upgraded product stream and stabilizer column off-gas stream, wherein at least a portion of said stabilizer column off-gas stream is arranged to be recycled as additional feed to the biomass digester.

Preferably, the stabilizer column off-gas stream and the vent column off-gas stream are arranged to be combined prior to being recycled as additional feed to the biomass digester.

As noted, the off-gas streams comprise hydrocarbons, and may advantageously be recycled. Therefore, according to the invention, at least a portion of the first and/or at least a portion of the second off-gas stream is arranged to be recycled as additional feed to the biomass digester.

In addition, at least a portion of the first and/or at least a portion of the second off-gas stream is arranged to be provided as heating fuel for one or more components of the plant.

### Process

The present technology also provides a process for producing a raw product stream from a first biomass feed, in a chemical plant as described herein. The process comprises the general steps of:
feeding a first biomass feed to a biomass digester, and converting it to a biogas stream,
feeding at least a portion of the biogas stream from the biomass digester to the reformer section so as to provide a first synthesis gas stream,
feeding a synthesis gas stream from the reformer section to the synthesis section so as to provide a raw product stream; and a first hydrocarbon-containing off-gas stream,
feeding at least a portion of the raw product stream to a distillation section so as to provide at least an upgraded product stream and a second hydrocarbon-containing off-gas stream, and;
recycling at least a portion of said first and/or at least a portion of said second off-gas stream as additional feed to the biomass digester.

Suitably, in said process the synthesis section is a methanol synthesis section and the raw product stream is a raw methanol stream.

All details of the plant described above are equally relevant for the process of the invention, mutatis mutandis.

The present invention has been described with reference to a number of embodiments and figures. However, the skilled person is able to select and combine various embodiments within the scope of the invention, which is defined by the appended claims. All documents mentioned herein are incorporated by reference.

### Example 1

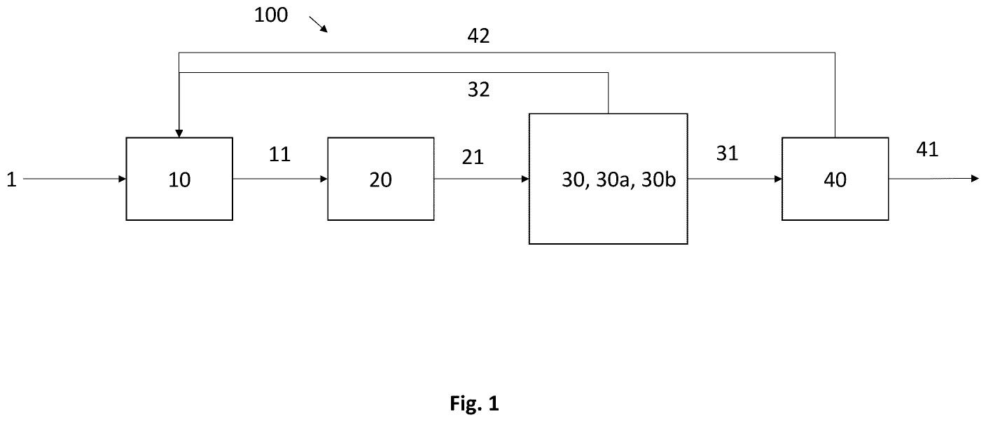

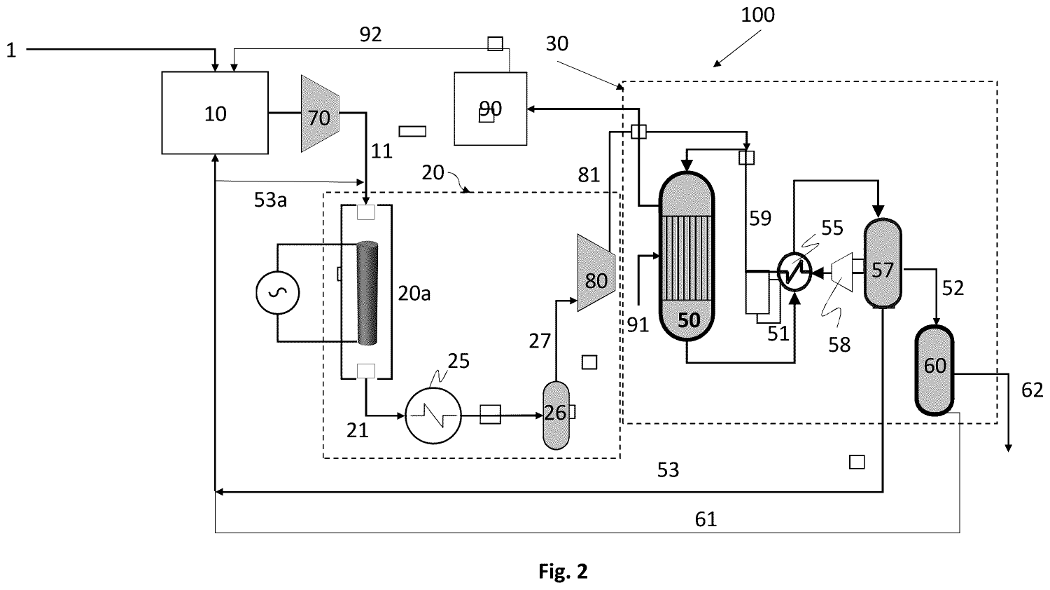

As illustrated in FIG. 1 and—in particular in FIG. 2—a methanol plant 100 is fed with biogas 11, from biomass digester 10. The biogas feed is 5000 Nm³/h biogas together with additional 193 Nm³/h biogas generated from recycled carbon. The biogas stream 11 is pretreated in a biogas upgrade unit removing a fraction of the CO₂ to meet the module for the methanol synthesis. The upgrade biogas is compressed to 25 barg and preheated to 350° C. A biogas compressor 70 compresses the biogas.

After mixing with 204 Nm³/h H₂ (recycled from the methanol unit) the biogas stream is led through a sulphur clean up unit consisting of two reactors (not shown in FIG. 2). Additional 853 Nm³/h H₂ (recycled from the methanol unit and 4158 kg/h steam is mixed with the desulphurized process stream. This process stream is heated to 450° C. and led to an adiabatic prereformer (not shown in FIG. 2). In the prereformer, the steam reforming reaction and the water gas shift reaction is equilibrated.

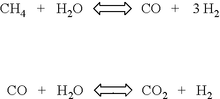

The prereformed process gas is led to the electrical heated reformer (20a), where synthesis gas stream (21) is produced. The synthesis gas leaves the reactor at 950° C. The produced synthesis gas is cooled in several heat exchangers (25) to form steam, to heat boiler feed water, to provide heat for the stabilizer column reboiler and preheat demineralised water before final cooling by cooling water to reach 40° C. The condensed water in the syngas is separated from the syngas in a separator (26) resulting in 2193 kg/h of process condensate.

The syngas stream (27) is compressed to 90 bar_{g} and mixed into the methanol synthesis loop after the recycle compressor (80). At the compression stage (80), an additional 23 kg/h process condensate (82) is formed. The mix of recycle gas and make-up syngas is preheated in a feed effluent (F/E) heat exchanger (55) to 220° C. and led through the methanol reactor (50). The methanol reactor (50) is a boiling water reactor generating a duty of 3.69 MW thermal heat resulting in an exit temperature of 249° C. of the converted syngas (51) being cooled in the F/E heat exchanger (55) to 114° C. Additional two heat exchangers cool the converted syngas to 40° C., which is then led to a high pressure separator (57) and separated into a liquid second methanol stream (52) and a gaseous recycle stream, this gaseous recycle stream is split into a syngas recycle stream (59) and a purge gas stream (53). The purge gas stream (53) from the high-pressure separator (57) is arranged to be recycled as hydrogen addition to the biogas feed (53a) and additional feed to the biomass digester (10).

After being reduced in pressure to 4 barg, the condensed methanol fraction is led to a low pressure separator (60) where an additional gaseous off-gas (61) is removed. The gaseous off-gas (61) is recycled to the biogas unit. The liquid methanol fraction (62) from the low pressure separator (60) is led to a raw methanol tank (150) in which the continuous fumes are washed with water (153). The water is mixed with the raw methanol in the tank, the washed gases (being another off gas stream (152)) are sent back to the biogas unit.

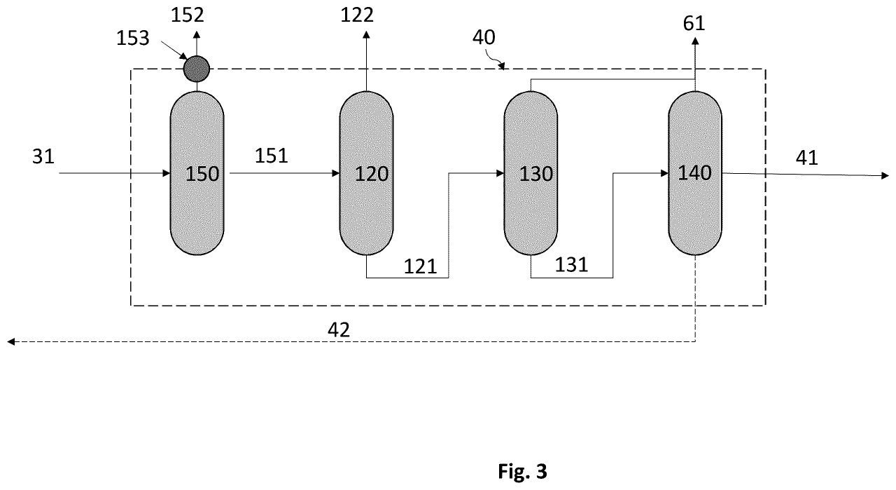

### Distillation Section 40—FIG. 3

The raw methanol stream (62) is pumped to a vent wash column (150). Vent wash column (150) is arranged to receive at least a portion of the raw product stream (31) from the synthesis section (30) and provide at least a first upgraded product stream (151) and a vent column off-gas stream (152). At least a portion of said vent column off-gas stream (152) is arranged to be recycled as additional feed to the biomass digester (10).

In a stabilizer column (120), additional off-gases (122) are evaporated from the raw methanol. The stabilizer column (120) is arranged to receive at least a portion of the first upgraded product stream (151) from the vent wash column (150), and provide at least a second upgraded product stream (121) and stabilizer column off-gas stream (122), wherein at least a portion of said stabilizer column off-gas stream (122) is arranged to be recycled as additional feed to the biomass digester (10).

The stabilized methanol stream (121) leaving the stabilizer column (120) is sent through two distillation columns (130, 140). The first distillation column (130) is at low pressure (ca. 0.8 barg) and the second (140) is at medium pressure (ca. 3.7 barg). Distilling the methanol product from species with higher boiling point also leads to a purge stream comprising higher alcohols and an excess water stream. The excess water stream is split into a wash water stream for the raw methanol tank (153) and a recycle stream to the biogas unit. The higher alcohol stream may also be recycled to the biogas unit.

1. A plant, said plant comprising:
a first biomass feed,
a biomass digester, configured to receive the first biomass feed and convert the first biomass feed to a biogas stream,
a reformer section configured to receive at least a portion of the biogas stream from the biomass digester and provide a first synthesis gas stream,
a synthesis section, configured to receive a synthesis gas stream from the reformer section, and to provide; a raw product stream; and a first hydrocarbon-containing off-gas stream,
a distillation section configured to receive at least a portion of the raw product stream and provide at least an upgraded product stream and a second hydrocarbon-containing off-gas stream,
wherein at least a portion of said first hydrocarbon-containing off-gas stream and/or at least a portion of said second hydrocarbon-containing off-gas stream is/are configured to be recycled as additional feed to the biomass digester.

2. The plant according to claim 1, wherein the synthesis section is a methanol synthesis section and the raw product stream is a raw methanol stream.

3. The plant according to claim 2, wherein the methanol synthesis section comprises:
a methanol reactor configured to receive the synthesis gas stream from the syngas section and provide a first methanol stream,
a high-pressure separator configured to receive the first methanol stream from the methanol reactor and separate the first methanol stream into a second methanol stream and a purge gas stream,
wherein the purge gas stream from the high-pressure separator is configured to be recycled as additional feed to the biomass digester.

4. The plant according to claim 3, wherein the methanol synthesis section further comprises a low-pressure separator configured to receive the second methanol stream from the high-pressure separator and provide a raw methanol stream and a third off-gas stream, and wherein the third off-gas stream is configured to be recycled as additional feed to the biomass digester.

5. The plant according to claim 1, wherein the synthesis section is a Fischer-Tropsch synthesis section, and the raw product stream is a raw hydrocarbon stream.

6. The plant according to claim 1, wherein the reformer section comprises an electrically heated Steam Methane Reformer.

7. The plant according to claim 1, wherein the distillation section comprises a vent wash column, wherein said vent wash column is configured to receive the at least a portion of the raw product stream from the synthesis section and provide at least a first upgraded product stream and a vent column off-gas stream, wherein at least a portion of said vent column off-gas stream is configured to be recycled as additional feed to the biomass digester.

8. The plant according to claim 7, wherein the distillation section comprises a stabilizer column, wherein said stabilizer column is configured to receive at least a portion of the first upgraded product stream from the vent wash column, and provide at least a second upgraded product stream and stabilizer column off-gas stream, wherein at least a portion of said stabilizer column off-gas stream is configured to be recycled as additional feed to the biomass digester.

9. The plant according to claim 8, wherein the stabilizer column off-gas stream and the vent column off-gas stream are configured to be combined prior to being recycled as additional feed to the biomass digester.

10. The plant according to claim 1, wherein at least a part of said first hydrocarbon-containing off-gas stream and/or at least a part of said second hydrocarbon-containing off-gas stream is/are configured to be provided as heating fuel for one or more components of the plant.

11. The plant according to claim 1, further comprising means for heating the biomass digester.

12. The plant according to claim 11, wherein at least a part of said first hydrocarbon-containing off-gas stream and/or at least a part of said second hydrocarbon-containing off-gas stream, or a combination of said first and second off-gas streams, is/are configured to be fed through said heat exchanger, thereby heating the biomass digester.

13. The plant according to claim 1, wherein the reformer section and/or the synthesis section comprise one or more heat exchangers, configured to exchange heat between one or more cooling streams in said plant and one or more streams in said reformer section and/or said synthesis section; and thus provide one or more heated streams from said cooling streams, and wherein at least a portion of said heated stream(s) is configured to heat the biomass digester.

14. A process for providing a product stream from a first biomass feed, in a plant according to claim 1, said process comprising:
feeding the first biomass feed to the biomass digester, and converting the first biomass feed to a biogas stream,
feeding at least a portion of the biogas stream from the biomass digester to the reformer section so as to provide a first synthesis gas stream,
feeding a synthesis gas stream from the reformer section to the synthesis section, so as to provide: a raw product stream; and a first hydrocarbon-containing off-gas stream,
feeding at least a portion of the raw product stream to the distillation section so as to provide at least an upgraded product stream and a second hydrocarbon-containing off-gas stream, and;
recycling at least a portion of said first hydrocarbon-containing off-gas stream and/or at least a portion of said second hydrocarbon-containing off-gas stream as additional feed to the biomass digester.

15. The process according to claim 14, wherein the synthesis section is a methanol synthesis section and the raw product stream is a raw methanol stream.

16. The plant according to claim 1, wherein the at least a portion of said first hydrocarbon-containing off-gas stream is configured to be recycled as additional feed to the biomass digester.

17. The plant according to claim 1, wherein the at least a portion of said second hydrocarbon-containing off-gas stream is configured to be recycled as additional feed to the biomass digester.

18. The plant according to claim 1, further comprising a heat exchanger configured for heating the biomass digester.
